(19) United States
(12) Patent Application Publication
(10) Pub. No.: US 2005/0266098 A1
(43) Pub. Date: Dec. 1, 2005

(54) Composition for treatment of a hydrofluoric acid burn

(76) Inventors: Gioulchen Tairova, Cobourg (CA); Engin Ozberk, Cobourg (CA); Noel Kerin, Toronto (CA)

Correspondence Address: WILMER CUTLER PICKERING HALE AND DORR LLP, 60 STATE STREET, BOSTON, MA, 02109, US

(21) Appl. No.: 11/141,904

(22) Filed: Jun. 1, 2005

Related U.S. Application Data
(60) Provisional application No. 60/575,397, filed on Jun. 1, 2004.

(51) Int. Cl. A61K 33/06; A61K 31/19

(52) U.S. Cl. 424/682; 514/557

(57) ABSTRACT

The present invention provides a composition for the treatment of a hydrofluoric acid burn. The composition comprises a non-toxic calcium compound, for example a non-toxic organic calcium compound such as calcium levulinate, in a carrier, such as water, an aqueous dimethyl sulphoxide solution, or an aqueous urea solution. When applied to an affected area, calcium ions react with fluoride ions, thereby neutralizing the fluoride ions and preventing further damage to the skin tissue in or on the affected area.

This application claims the benefit of U.S. Provisional Application No. 60/575,397, filed Jun. 1, 2004.

### FIELD OF THE INVENTION

This invention relates to a composition comprising a non-toxic calcium compound and a carrier for the treatment of a hydrofluoric acid burn. In a preferred embodiment, the invention relates to a calcium levulinate composition for treatment of a hydrofluoric acid burn.

### BACKGROUND OF THE INVENTION

It is well known that strong acids, especially concentrated inorganic acids, can attack skin [1]. In general, acid contact with skin results in localized irritation, but an appreciable number of acids, such as hydrofluoric acid, are absorbed through the skin and can produce systemic poisoning.

Hydrofluoric acid is an inorganic acid which may cause skin/tissue/flesh damage via two mechanisms: (1) corrosive burns due to free hydrogen ions, and (2) chemical burns from tissue penetration by fluoride ions. In the latter case, the main portals of entry for hydrofluoric acid through the skin are hair follicles, sebaceous glands, sweat glands and cuts or abrasions of the outer layers of the skin. There are numerous blood vessels immediately below the skin, which facilitate the absorption of chemicals for transport throughout the entire body.

The most deleterious injuries arise when fluoride ions from the hydrofluoric acid bind with calcium ions in human cellular materials. This scavenging of calcium ions may lead to the condition described as hypocalcaemia and/or cellular necrosis and death. This process, if untreated, can continue for several days, causing increased tissue damage. Even after thorough washing of the exposed skin with large amounts of water, the hydrofluoric acid molecules under the skin may continue to enter into other parts of the body and cause tissue destruction. Hydrofluoric acid possesses an unusual penetrating ability that requires immediate and proper treatment to prevent further damage.

In addition to washing with water, the most effective way to prevent or slow down the effects of hydrofluoric acid penetration into the tissue is to neutralize the fluoride ions as quickly as possible to prevent further reaction of the fluoride ions with tissue in/on or under the exposed area. This can be done either by complexation of the fluoride ions or by delivering a high concentration of active ions, such as Ca²⁺, which react with fluoride ions to form CaF₂ which can then be removed from the damaged area.

Currently, the most widely used treatment of hydrofluoric acid injuries comprises a benzalkonium chloride such as [C₆H₅CH₂N(CH₃)₂R]⁺Cl⁻ where R represents a mixture of alkyls from C₈H₁₇ to C₁₈H₃₇) and solutions of calcium gluconate, which are applied either as a topical application or as an injection [2-10].

The major problem associated with the use of the quaternary ammonium compounds, Hyamine® and Zephiran®, is their toxicity. For example, it is estimated that 1-3 g of Hyamine could be fatal [3]. This dosage is equivalent to that supplied in 50-150 mL of a 2 wt % solution. Other disadvantages of quaternary ammonium compounds are:
(i) the solutions are applied at ice-water temperature often causing patients to experience discomfort;
(ii) it is a time-consuming procedure to change compresses every few minutes for the several hours recommended; and
(iii) compresses often cause painful reaction when applied on a patient's head, neck or near the mucous membranes.

Toxicology and carcinogenesis studies of Hyamine® were conducted on rats and mice by the National Cancer Institute [11]. The research indicated that exposure of rats and mice to Hyamine® led to lesions (16 days) and ulcers (13 weeks) at the site of application.

The major disadvantages associated with the calcium gluconate treatment are:
(i) calcium gluconate is not very soluble in water (0.4 g/100 mL at 20° C.) and forms unstable suspensions in the absence of an organic stabilizer and aqueous hydrochloric acid;
(ii) when applied topically, calcium gluconate solutions are often irritating especially when used for the treatment of eyes [2];
(iii) calcium gluconate injections for deep penetrating burns;
(iv) injections are often painful and there is the possibility of infection or tissue necrosis especially when used on digits [2, 3, 6-10]; and
(v) in some cases, injections are ineffective [4].

Calcium gluconate 10% (wt/wt) in dimethyl sulphoxide (DMSO) has also been used for treatment of hydrofluoric acid burns on laboratory rats [12, 13]. The efficacy of these treatments may have been limited by the low solubility of calcium gluconate.

Calcium acetate soaks were also tested as a source of calcium ions for treatment of hydrofluoric acid injury [4]. Their major disadvantage is a negative epidermal response that results from the treatment.

Additional compositions for the treatment of hydrofluoric acid burns are described in the French patent No. FR2604900 issued 15 Apr. 1988 to M. C. Blomet and entitled “Physiological Solution for Washing Parts of the Human Body Which Have Come into Contact with Hydrofluoric Acid and Concentrate for Preparing It” [14]. The active ingredients disclosed therein are ethylenediaminetetraacetate tetrasodium salt and aluminum nitrate combined in various ratios. The major disadvantage of application of these compositions arises from the fact that ethylenediaminetetraacetate tetrasodium salt is a cancer suspect and an irritant [15]. A further disadvantage of using these mixtures is that they can only be applied for decontamination (washing) of hydrofluoric acid and are not suitable for the treatment of delayed or deeper burns.

The composition called Hexafluorine®, produced by laboratoire PREVOR in France, is described as an effective treatment for immediate decontamination (washing) of HF exposed eye/skin [16]. The major disadvantage of using Hexafluorine® arises from the fact that it is not suitable for the treatment of delayed or deeper burns.

### SUMMARY OF THE INVENTION

The present invention provides a composition for the treatment of a hydrofluoric acid burn. The composition comprises a non-toxic calcium compound, for example a non-toxic organic calcium compound such as calcium levulinate, and a carrier, for example water, an aqueous dimethyl sulphoxide solution, or an aqueous urea solution, which is applied to an affected area. The composition provides for the delivery of calcium ions to the affected area to neutralize fluoride ions and prevent further damage to the affected area. The composition also provides for trapping of free hydrogen ions thus preventing further corrosive damage to the affected area. Consequently, the compositions described herein may be used in the treatment of corrosive burns not only from hydrofluoric acid, but also from other inorganic acids.

The composition has particular importance in the treatment of superficial and/or delayed or deeper hydrofluoric acid burns through the rapid delivery of calcium ions to the affected area.

According to one aspect of the present invention, there is provided a composition for the treatment of a hydrofluoric acid burn, comprising a non-toxic calcium compound having an aqueous solubility of at least 10 g/100 mL at room temperature and a carrier.

According to a further aspect of the present invention, the non-toxic calcium compound is an organic calcium compound. An organic calcium compound is a calcium compound having a counterion comprising carbon-hydrogen bonds.

According to another aspect of the present invention, there is provided a composition for the treatment of a hydrofluoric acid burn comprising calcium levulinate and a carrier.

According to yet another aspect of the present invention, there is provided a method of treating a hydrofluoric acid burn comprising administering a therapeutically effective amount of a composition as defined herein to a patient or subject in need thereof.

According to still another aspect of the present invention, there is provided a method of treating a hydrofluoric acid burn comprising administering a therapeutically effective amount of calcium levulinate to a patient or subject in need thereof.

According to a further aspect of the present invention, there is provided a use of a composition, as defined herein, for the treatment of a hydrofluoric acid burn.

According to another aspect of the present invention, there is provided a use of a composition, as defined herein, for the manufacture of a medicament for the treatment of a hydrofluoric acid burn.

According to yet another aspect of the present invention, there is provided a use of calcium levulinate for the treatment of a hydrofluoric acid burn.

According to still another aspect of the present invention, there is provided a use of calcium levulinate for the manufacture of a medicament for the treatment of a hydrofluoric acid burn.

According to a further another aspect of the present invention, there is provided a commercial package comprising a composition, as defined herein, together with instructions for its use in treating a hydrofluoric acid burn.

According to another aspect of the present invention, there is provided a commercial package comprising calcium levulinate together with instructions for its use in treating a hydrofluoric acid burn.

### DETAILED DESCRIPTION OF THE INVENTION

There is provided a use of a non-toxic calcium compound for the treatment of a hydrofluoric acid burn. The term “non-toxic” refers to a calcium compound in which any toxic or detrimental effects of the calcium compound are outweighed by the therapeutically beneficial effects. Preferably, the non-toxic calcium compound has an aqueous solubility of at least 10 g/100 mL at room temperature, more preferably an aqueous solubility of at least 20 g/100 mL at room temperature, and most preferably an aqueous solubility of at least 30 g/100 mL at room temperature. The non-toxic calcium compound may be an organic calcium compound. An example of a non-toxic organic calcium compound is calcium levulinate.

The non-toxic calcium compound may be used together with a carrier in a composition for the treatment of a hydrofluoric acid burn. The composition comprises the non-toxic calcium compound preferably at a concentration of 10 to 40 wt %, more preferably at a concentration of 15 to 35 wt %, and most preferably at a concentration of 20 to 30 wt %.

The carrier is also non-toxic in that any toxic or detrimental effects of the carrier are outweighed by the therapeutically beneficial effects. The carrier also has the property of skin permeability, which permits the non-toxic calcium compound to penetrate the skin barrier.

The carrier may be water, an aqueous dimethyl sulfoxide solution, or an aqueous urea solution. Preferably, the aqueous dimethyl sulfoxide solution is at a concentration of 10 to 45 wt %, more preferably at a concentration of 15 to 40 wt %, and most preferably at a concentration of 20 to 35 wt %. Preferably, the aqueous urea solution is at a concentration of 5 to 25 wt % and more preferably at a concentration of 10 to 20 wt %.

A person skilled in the art would recognize that the carrier may also be selected from polyethylene glycol monolaurate, eucalyptol, a halogenated compound selected from trichloroethanol and trifluoroethanol, a lanoline derivative, a 1-substituted azacycloalkan-2-one, a urethane compound, polyvinyl-pyrrolidone, a binary composition of N-(2-hydroxyethyl)-pyrrolidone and methyl laureate or oleic acid or oleyl alcohol, and a pyrrolidone-type compound.

In various embodiments, a non-toxic calcium compound, e.g. a non-toxic organic calcium compound such as calcium levulinate may be used therapeutically in formulations or medicaments to treat a hydrofluoric acid burn. The invention provides corresponding methods of medical treatment, in which a therapeutic dose of a non-toxic calcium compound is administered in a pharmacologically acceptable formulation, e.g. to a patient or subject in need thereof. Accordingly, the invention also provides therapeutic compositions comprising a non-toxic calcium compound, e.g. a non-toxic organic calcium compound such as calcium levulinate, and a pharmacologically acceptable excipient or carrier. In one embodiment, such compositions include a non-toxic calcium compound in a therapeutically effective amount sufficient to treat a hydrofluoric acid burn.

A “therapeutically effective amount” refers to an amount effective, at dosages and for periods of time necessary, to achieve the desired therapeutic result, such as a reduction of tissue damage. A therapeutically effective amount of a non-toxic calcium compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects.

Therapeutic compositions typically must be sterile and stable under the conditions of manufacture and storage. The composition can be formulated as a solution. In many cases, it will be preferable to include isotonic agents, for example, sugars, polyalcohols such as mannitol, sorbitol, or sodium chloride in the composition. Prolonged absorption of the compositions can be brought about by including in the composition an agent which delays absorption, for example, monostearate salts and gelatin. Moreover, a non-toxic calcium compound can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile solutions can be prepared by incorporating the active compound (e.g. a non-toxic calcium compound) in the required amount in an appropriate solvent with one or a combination of ingredients enumerated above, as required, followed by filtered sterilization. In the case of sterile powders for the preparation of sterile solutions, the preferred methods of preparation are vacuum drying and freeze-drying which yields a powder of the active ingredient plus any additional desired ingredient from a previously sterile-filtered solution thereof. In accordance with an alternative aspect of the invention, a non-toxic calcium compound may be formulated with one or more additional compounds that enhance the solubility of the non-toxic calcium compound.

A non-toxic calcium compound, for example a non-toxic organic calcium compound such as calcium levulinate may be provided in containers or commercial packages which further comprise instructions for use of the non-toxic calcium compound for the treatment of a hydrofluoric acid burn. Further, a composition comprising a non-toxic calcium compound, for example a non-toxic organic calcium compound such as calcium levulinate and a carrier may be provided in containers or commercial packages which further comprise instructions for use of the composition for the treatment of a hydrofluoric acid burn.

In a preferred embodiment, the composition contains calcium levulinate and a carrier for the rapid delivery of calcium ions to fluoride-exposed tissue. Interpretation of the studies described below suggest that calcium ions reacted with fluoride ions to form non-toxic calcium fluoride, as in equation [a]:
(CH₃COCH₂CH₂COO)₂Ca+2HF→CH₃COCH₂CH₂COOH+CaF₂ [a]

The calcium fluoride product was identified by X-ray diffraction (XRD) analyses. In the experiments carried out, the calcium fluoride was precipitated as fine particles (particle size<0.2 μm).

The other product of the reactions was levulinic acid, which was identified by Raman spectroscopy. Levulinic acid, a weak non-toxic acid (for 98% aqueous solution pH is 6.2 at 18° C.), is found in many fruits and vegetables and is widely used as an additive in the food industry. Levulinic acid has a low dissociation constant (pKₐ=4.64 at 18° C. [17]) and binds hydronium ions, H₃O⁺, according to the equation [b]:
CH₃COCH₂CH₂COOH+H₂O⇄CH₃COCH₂CH₂COO⁻+H₃O⁺[b]

Surprisingly, Applicants have determined that a composition containing a non-toxic calcium compound having an aqueous solubility of at least 10 g/100 mL, for example a non-toxic organic calcium compound such as calcium levulinate, can be applied to an affected area and effectively remove fluoride ions to prevent further reaction of fluoride ion with skin tissue in or on the affected area. The non-toxic calcium compound, such as the calcium levulinate contained in the compositions described in the examples, binds both F⁻ and H₃O+, apparently as described previously in equations [a]and [b].

It is desirable to select a calcium compound that is non-toxic and causes less irritation to the skin than other calcium compounds [18]. Further, the non-toxic calcium compound should be soluble in aqueous solution, and preferably in aqueous solution with a carrier such as DMSO or urea. For example, calcium levulinate has been found to exhibit favourable solubility in water and in aqueous solution with a carrier such as DMSO or urea. The examples described below illustrate that calcium levulinate is more soluble, in different aqueous solvent systems at several temperatures, than the calcium gluconate used in existing commercial compositions. Therefore, a composition comprising calcium levulinate may deliver a potentially higher concentration of fluoride sequestering agent to the affected area than prior art compositions comprising calcium gluconate.

The membrane-penetrating ability of a carrier such as DMSO may enhance absorption of the non-toxic calcium compound into skin tissue. This enhanced absorption may lead to delivery of calcium ions deeper into an affected area than use of a non-toxic calcium compound without a carrier. As a result, fluoride ions that have penetrated into skin tissue may be neutralized thereby limiting or preventing extensive tissue damage due to delayed or deeper HF burns.

Preferably, the pH range during treatment of HF burns with a non-toxic calcium compound is maintained between 3.4 and 6.5, which is physiologically reasonable and acceptable [19]. The pH could be maintained closer to physiological pH 7.4, if large excess (as compared to the stoichiometric amount of a non-toxic calcium compound required for the reaction with a given amount of hydrofluoric acid) of the composition comprising the non-toxic calcium compound is applied. As illustrated in the examples, a composition comprising a non-toxic calcium compound such as calcium levulinate and a carrier maintains the pH in the range of 3.4 to 5.8 during reaction with hydrofluoric acid.

### EXAMPLES

The following examples are provided to illustrate the invention. It will be understood, however, that the specific details given in each example have been selected for the purpose of illustration and are not to be construed as limiting in scope of the invention.

In Example 1, the solubility of calcium levulinate is compared to prior art calcium gluconate. The reactions of various calcium levulinate compositions with hydrofluoric acid are illustrated in Examples 2-8, in all of which concentrations are expressed in weight % (wt %).

### Example 1

### Solubility of Calcium Levulinate

Solubility studies of calcium gluconate and calcium levulinate in various solvent systems at temperatures 20° C., 37° C., 60° C. and 90° C. are given in Table 1. The results show calcium levulinate to be more soluble than calcium gluconate in the listed solvent systems.

TABLE 1
Solubilities of active compounds (g/100 mL of
solvent) at various temperatures

| | T | Calcium gluconate | Calcium levulinate |
|---|---|---|---|
| Solvent | ° C. | g/100 mL | g/100 mL |
| Water | 20 | 0.4 | 38.0 |
| | 37 | 1.0 | 48.1 |
| | 60 | 2.4 | 55.2 |
| | 90 | 5.6 | 90.0 |
| 45% DMSO in water | 20 | 0.2 | 14.0 |
| | 37 | 0.4 | 15.0 |
| | 60 | 1.0 | 20.1 |
| | 90 | 1.3 | 44.6 |
| 10% urea in water | 20 | 0.4 | 38.1 |
| | 37 | 1.0 | 47.1 |
| | 60 | 1.4 | 55.1 |
| | 90 | 2.2 | 74.3 |
| 100% DMSO | 20 | 0.1 | 42.8 |
| | 37 | 0.3 | 58.4 |
| | 60 | 0.6 | 70.0 |
| | 90 | 1.1 | 92.3 |

### Example 2

### Reaction of a Solution of 10% Calcium Levulinate in Water with an Equal Molar Solution of 48% Hydrofluoric Acid—[Calcium Levulinate]:[HF]=1:1.

5 g of calcium levulinate powder were dissolved in 45 ml of deionized water in a 150 mL beaker and placed into a water bath maintained at 37° C. The pH of the solution was 8.1.

1.5 g of 48% hydrofluoric acid was added to the beaker and the mixture stirred by means of a magnetic stirrer. Analysis of a 5 mL sample of the reaction solution taken five minutes after the start of the reaction indicated that 96% of the hydrofluoric acid had reacted during the first five minutes of the test and that the pH of the solution had dropped to 3.4.

### Example 3

### Reaction of a Solution of 20% Calcium Levulinate in Water with 48% Hydrofluoric Acid—[Calcium Levulinate]:[HF]=2:1.

10 g of calcium levulinate powder were dissolved in 40 mL of deionized water in a 150 mL beaker and placed into a water bath maintained at 37° C. The pH of the solution was 8.1.

1.5 g of 48% hydrofluoric acid was added to the beaker and the mixture stirred by means of a magnetic stirrer. Analysis of a 5 mL sample of the reaction solution taken five minutes after the start of the reaction indicated that 97% of the hydrofluoric acid had reacted during the first five minutes of the test and that the pH of the solution had dropped to 4.4.

### Example 4

### Reaction of a Solution of 30% Calcium Levulinate in Water with 48% Hydrofluoric Acid—[Calcium Levulinate]:[HF]=3:1.

15 g of calcium levulinate powder were dissolved in 35 mL of deionized water in a 150 mL beaker and placed into a water bath maintained at 37° C. The pH of the solution was 8.4.

1.5 g of 48% hydrofluoric acid was added to the beaker and the mixture stirred by means of a magnetic stirrer. Analysis of a 5 mL sample of the reaction solution taken five minutes after the start of the reaction indicated that 92% of the hydrofluoric acid had reacted during the first five minutes of the test and that the pH of the solution had dropped to 4.7.

### Example 5

### Reaction of a Solution of 10% Calcium Levulinate, 10% Urea and 80% Water with 48% Hydrofluoric Acid—[Calcium Levulinate]:[HF]=1:1.

5 g calcium levulinate and 5 g urea were dissolved in 40 mL deionized water in a 150 mL beaker inside a water bath maintained at 37° C. The pH of the solution was 8.2.

1.5 g of 48% hydrofluoric was added to the beaker and the mixture stirred by means of a magnetic stirrer. Analysis of a 5 mL sample of the reaction solution taken five minutes after the start of the reaction indicated that 92% of the hydrofluoric acid had reacted during the first five minutes of the test and that the pH of the solution had dropped to 4.2.

### Example 6

### Reaction of a Solution of 10% Calcium Levulinate in 10% Urea and 80% Water with 48% Hydrofluoric Acid—[Calcium Levulinate]:[HF]=2:1.

5 g calcium levulinate and 5 g urea were dissolved in 40 mL of deionized water in a 150 mL beaker and placed into a water bath maintained at 37° C. The pH of the solution was 8.2.

0.75 g of 48% hydrofluoric acid was added to the beaker and the mixture stirred by means of a magnetic stirrer. Analysis of a 5 mL sample of the reaction solution taken five minutes after the start of the reaction indicated that 93% of the hydrofluoric acid had reacted during the first five minutes of the test and that the pH of the solution had dropped to 4.6.

### Example 7

### Reaction of a Solution of 10% Calcium Levulinate in 45% DMSO and 45% Water with 48% Hydrofluoric Acid—[calcium levulinate]:[HF]=1:1.

5 g calcium levulinate were dissolved in a solution of 22.5 g DMSO and 22.5 mL of deionized water in a 150 mL beaker and placed into a water bath maintained at 37° C. The pH of the solution was 9.2.

1.5 g of 48% hydrofluoric acid was added to the beaker and the mixture stirred by means of a magnetic stirrer. Analysis of a 5 mL sample of the reaction solution taken five minutes after the start of the reaction indicated that 92% of the hydrofluoric acid had reacted during the first five minutes of the test and that the pH of the solution had dropped to 4.4.

### Example 8

### Reaction of a Solution of 10% Calcium Levulinate in 45% DMSO and 45% Water with 48% Hydrofluoric Acid—[Calcium Levulinate]: [HF]=2:1.

5 g of calcium levulinate were dissolved in a solution of 22.5 g DMSO and 22.5 mL of deionized water in a 150 mL beaker and placed into a water bath maintained at 37° C. The pH of the solution was 9.2.

0.75 g of 48% hydrofluoric acid was added to the beaker and the mixture stirred by means of a magnetic stirrer. Analysis of a 5 mL sample of the reaction solution was taken five minutes after the start of the reaction indicated that 83% of the hydrofluoric acid had reacted during the first five minutes of the test and that the pH of the solution had dropped to 5.8.

While embodiments of the present invention have been described in the foregoing, it is to be understood that other embodiments are possible within the scope of the invention. The invention is to be considered limited solely by the scope of the appended claims.

### REFERENCES

[1] Committee on Hazardous Substances in the Laboratory, National Research Council, “Prudent Practices for Handling Hazardous Chemicals in Laboratories”, National Academy Press, Washington, D.C., U.S.A., 1981, 33.
[2] M. A. MacKinnon, “Hydrofluoric Acid Burns”, Dermatologic Clinics, 1988, Vol. 6, No. 1, 67-74.
[3] J. J. R. Kirkpatrick, D. S. Enion and D. A. R. Burd, “Hydrofluoric Acid Burns: A Review”, Burns, 1995, Vol. 21, No. 7, 483-493.
[4] B. J. Dunn, M. A. MacKinnon, N. F. Knowlden, D. J. Billmaier, M. J. Derelanko, G. M. Rusch, D. J. Naas and R. R. Dahlgren, “Hydrofluoric Acid Dermal Burns. An Assessment of Efficacy Using an Experimental Pig Model”, J. Occup. Med., 1992, Vol. 34, No. 9, 902-909.
[5] B. J. Dunn, M. A. MacKinnon, N. F. Knowlden, D. J. Billmaier, M. J. Derelanko, G. M. Rusch, D. J. Naas and R. R. Dahlgren, “Topical Treatments for Hydrofluoric Acid Dermal Burns. Further Assessment of Efficacy Using an Experimental Pig Model”, J. Occup. Environ. Med., 1996, Vol. 38, No. 5, 507-514.
[6] F. Edelman, “Hydrofluoric Acid Burns”, J. Occup. Med., 1986, Vol. 1, No. 1, 89-103.
[7] D. Peters and R. Miethchen, “Symptoms and Treatments of Hydrogen Fluoride Injuries”, J. Fluorine Chem., 1996, No. 79, 161-165.
[8] S. W. Shewmake and B. G. Anderson, “Hydrofluoric Acid Burns”, Arch. Dermatol., 1979, Vol. 115, 593-596.
[9] M. S. El Saadi, A. H. Hall, P. K. Hall, B. S. Riggs, W. L. Augenstein and B. H. Rumack, “Hydrofluoric Acid Dermal Exposure”, Vet. Hum. Toxicol., 1989, Vol. 31, No. 3, 243-247.
[10] A. T. Jones, “The Treatment of Hydrofluoric Acid Burns”, J. Industrial Hygiene and Toxicology, 1939, Vol. 21, No. 6, 205-212.
[11] Toxicology and Carcinogenesis Studies of Benzethonium Chloride (Hyamine, CAS No. 121-54-0) in F344/N Rats and B6C3F1 Mice (Dermal Studies), NTIS Report No. PB96-162300, July 1995.
[12] L. S. Zachary, W. Reus, J. Gottieb, J. P. Heggers and M. C. Robson, “Treatment of Experimental Hydrofluoric Acid Burns”, J. Burn Care and Rehabilitation, 1986, Vol. 7, No. 1, 35-39.
[13] S. T. Seyb, L. Noordhock, S. Botens and M. M. Mani, “A Study to Determine the Efficacy of Treatment for Hydrofluoric Acid Burns”, J. Burn Care and Rehabilitation, 1995, Vol. 16, No. 3, 253-257.
[14] M. C. Blomet, “Physiological Solution for Washing Parts of the Human Body Which Have Come into Contact with Hydrofluoric Acid and Concentrate For Preparing It”, French Patent No. 2604900, 15 Apr. 1988.
[15] Sigma Aldrich Fine Chemicals, Aldrich Catalogue Handbook of Fine Chemicals, 1996, 686.
[16] A. H. Hall, J. Blomet, M. Gross and J. Nehles, “Hexafluorine for Emergent Decontamination of Hydrofluoric Acid Splashes”, SSA Journal, 2000, Vol. 14, 30-33.
[17] SRC Physical Properties Database, CAS No 000123 76-2.
[18] G. L. Jenkins, W. H. Hartung, K. E. Hamlin and J. B. Data, “The Chemistry of Organic Medicinal Products”, New York, John Wiley & Sons, 1957, 515.
[19] Improvements in or Relating to Chymotrypsin Solutions, Great Britain Patent No. 866423, 26 Apr. 1961.

1. A composition for the treatment of a hydrofluoric acid burn, comprising:
a non-toxic calcium compound having an aqueous solubility of at least 10 g/100 mL at room temperature; and
a carrier.

2. The composition according to claim 1, wherein the non-toxic calcium compound is an organic calcium compound.

3. The composition according to claim 1, wherein the aqueous solubility is at least 20 g/100 mL.

4. The composition according to claim 1, wherein the aqueous solubility is at least 30 g/100 mL.

5. The composition according to claim 1, wherein the non-toxic calcium compound is at a concentration of 10 to 40 wt %.

6. The composition according to claim 1, wherein the non-toxic calcium compound is at a concentration of 15 to 35 wt %.

7. The composition according to claim 1, wherein the non-toxic calcium compound is at a concentration of 20 to 30 wt %.

8. The composition according to claim 4, wherein the non-toxic calcium compound is calcium levulinate.

9. The composition according to claim 1, wherein the carrier is water, an aqueous dimethyl sulfoxide solution, or an aqueous urea solution.

10. The composition according to claim 9, wherein the aqueous dimethyl sulfoxide solution is at a concentration of 10 to 45 wt %.

11. The composition according to claim 9, wherein the aqueous dimethyl sulfoxide solution is at a concentration of 15 to 40 wt %.

12. The composition according to claim 9, wherein the aqueous dimethyl sulfoxide solution is at a concentration of 20 to 35 wt %.

13. The composition according to claim 9, wherein the aqueous urea solution is at a concentration of 5 to 25 wt %.

14. The composition according to claim 9, wherein the aqueous urea solution is at a concentration of 10 to 20 wt %.

15. The composition according to claim 1, wherein the carrier is polyethylene glycol monolaurate, eucalyptol, a halogenated compound selected from trichloroethanol and trifluoroethanol, a lanoline derivative, a 1-substituted azacycloalkan-2-one, a urethane compound, polyvinylpyrrolidone, a binary composition of N-(2-hydroxyethyl)pyrrolidone and methyl laureate or oleic acid or oleyl alcohol, or a pyrrolidone-type compound.

16. A method of treating a hydrofluoric acid burn comprising administering a therapeutically effective amount of a composition as defined in claim 1 to a patient or subject in need thereof.

17. A method of treating a hydrofluoric acid burn comprising administering a therapeutically effective amount of calcium levulinate to a patient or subject in need thereof.

18. A commercial package comprising a composition as defined in claim 1 together with instructions for its use in treating a hydrofluoric acid burn.

19. A commercial package comprising calcium levulinate together with instructions for its use in treating a hydrofluoric acid burn.
